(19) United States
(12) Patent Application Publication
(10) Pub. No.: US 2004/0220604 A1
(43) Pub. Date: Nov. 4, 2004

(54) Tissue separation apparatus and method

(76) Inventors: Thomas J. Fogarty, Portola Valley, CA (US); D. Bruce Modesitt, San Carlos, CA (US); Neil Holmgren, Alameda, CA (US)

Correspondence Address: David A. Levine, P.O. Box 61180, Palo Alto, CA, 94306, US

(21) Appl. No.: 10/426,983

(22) Filed: Apr. 30, 2003

(51) Int. Cl. A61B 17/00

(52) U.S. Cl. 606/190

(57) ABSTRACT

A tissue separation apparatus is disclosed. The apparatus can have a separating member that has a contracted state and an expanded state. When the separating member is placed between first and second tissue layers, the separating member can part the first tissue layer from the second tissue layer. The separating member can be moved into the contracted state to sever the first tissue layer. Methods of making and using the prosthesis are also disclosed.

### BACKGROUND OF THE INVENTION

1. Field of the Invention The present invention relates generally to a biological tissue separation device and methods of using the same.

2. Description of the Related Art

Diseased blood vessels can form an occlusion in the central channel of the vessel. The occlusion is formed from soft thrombus, hard, calcified plaque, or a combination thereof. The shape of the occlusions can also vary. For example, the occlusions can be total occlusions, flush occlusions or a combination thereof.

The inner inner vascular layer(s) (e.g., intima and sometimes media) of the diseased vessels can degrade badly enough to form a substantially homogenous form with the occlusion and can essentially eliminate a distinction between the vessel walls. The degraded inner vascular layer(s) combined with the occlusion form the diseased vascular core. Diseased vessels also often form a naturally weakened cleaving plane between the inner vascular layer(s) and the outer vascular layer(s) (e.g., adventitia and sometimes media).

Present endarterectomy methods use a blunt dissection technique to separate the core from the surrounding tissue, and some devices are available to sever the distal end of a diseased vessel core to remove it from the vascular bed. The vascular media is split from the intima or adventitia by forcing the tool directly into the cleaving plane. After cleaving the inner vascular layer from the outer vascular layer, the core of tissue including the occluded distal end of the intima is then severed and the diseased intima is pulled out of the body. Endarterectomy methods are described in Wesley S. Moore and Samuel S. Ahn, Endovascular Surgery, (3d ed. 2001) (for example at pp. 311-15) and U.S. Pat. No. 5,843,102 to Kalmann et al., both of the aforementioned publications are hereby incorporated by reference in their entireties.

One current endarterectomy process calls for the use of two devices. One device is used to cleave the length of intima from the adventitia. The second device is used to sever the distal end of the vascular core so the vascular core can be slid out from the adventitia and surrounding vascular bed.

U.S. Pat. No. 6,241,745 to Rosenthal discloses an endarterectomy instrument having a separation ring and a wire loop at the distal end of the instrument. The separation ring provides physical pressure to loosen the blockage and inner layer of the vessel from the outer layer of the vessel. Electrical current is passed through the wire loop to heat up the wire and help separate the inner layer of the vessel from the outer layer of the vessel. The wire is also electrically heated to cleanly sever the end of the inner layer of the vessel. Once the wire has cooled, the wire can also be used to snare and remove the severed end of the inner layer.

U.S. Pat. No. 6,517,550 to Kónya et al. discloses a retrieval device having a catheter with a lumen and a wire housed within the lumen. A portion of the wire is in the form of a loop and can be outside the catheter. The looped portion of the wire exits the catheter through a first opening in the catheter and enters the catheter through a second opening in the catheter. The first and second openings are positioned substantially equidistant from the distal end of the catheter. The wire is manipulated to open and close the loop. The retrieval device is used as a snare for grasping foreign articles and retrieving them from the body of the patient.

U.S. Pat. No. 5,133,725 to Quadri discloses a valvulotome having cutting blades and a hollow tube. A wire is slidably disposed in the tube, and the wire forms into several wire elements joined at a common point. Each cutting blade is mounted on a flat, spring-loaded wire element. During use, the wire element increases the longitudinal distance between the blade and the tube and the radial distance between different blades. When the wire is retracted, the blades also retract and cut the valve cusps

There is a need for a tissue separation apparatus that can diametrically expand and contract. There is also a need for an apparatus and method that can controllably separate one layer of tissue from an adjacent layer of tissue with minimal damage to the tissue layers. Furthermore, there is a need for an improved device that can cleave the plane and remove the diseased vascular core to assist in removal of the diseased vascular core from the outer vascular layer(s). There is also a need for a device that can address different types of occlusions in the same vessel (e.g., both flush and partial occlusions).

### BRIEF SUMMARY OF THE INVENTION

One aspect of the disclosed device is a tissue separating apparatus having a shaft and a first separating member. The shaft has a first end, a second end and a longitudinal axis. The first separating member is located at a first length along the longitudinal axis and at first angle from the longitudinal axis. The first separating member has a contracted state and an expanded state. When placed between a first tissue layer and a second tissue layer, the first separating member parts the first tissue layer from the second tissue layer in the expanded state and severs the first tissue layer in the contracted state.

The first separating member can also have a diameter from the longitudinal axis of the shaft. The diameter can vary during use of the apparatus. The diameter during the contracted state can be a second diameter, and the diameter during the expanded state can be a first diameter and can be larger than the second diameter. The first separating member can seat into the shaft when the first separating member is in the contracted state in a substantially linear fashion.

The first separating member in the expanded state can form a substantially helical shape, a substantially parabolic shape or a curved multi-parabolic shape. The first separating member can have a wire and a garotte.

One aspect of the disclosed tissue separation method includes separating a first tissue layer from a second tissue layer with a member, and severing the first tissue layer with the member. The member has an expanded state and a contracted state. Severing includes contracting the member from the expanded state into the contracted state.

Severing can also include moving a garotte through the first tissue layer. The member can also be inserted between the first tissue and second tissue layer. Separating can include contracting and expanding the member. Contracting the member can include actively or passively contracting the member. Expanding the member can also include actively or passively expanding the member.

### BRIEF DESCRIPTION OF THE DRAWINGS

FIG. 1 illustrates an embodiment of the tissue separation apparatus.

FIG. 2 illustrates an embodiment of the distal shaft end having the wire in an expanded configuration.

FIG. 3 is a cross-sectional view of an embodiment of the distal shaft end having the wire in an expanded configuration.

FIG. 4 illustrates an embodiment of the distal shaft end having the wire in an expanded configuration.

FIG. 5 is a perspective view of an embodiment of the distal shaft end having the wire in an expanded configuration.

FIG. 6 is a front view of an embodiment of the distal shaft end having the wire in an expanded configuration.

FIG. 7 is a cut-away view of an embodiment of the handle having the wire in an expanded configuration.

FIG. 8 illustrates the embodiment of FIG. 2 having the wire in a contracted configuration.

FIGS. 9-12 respectively illustrate the embodiments of FIG. 4-7 having the wire in contracted configurations.

FIG. 13 is a perspective view of an embodiment of the distal shaft end having the wire in an extended configuration.

FIG. 14 is a front view of the embodiment of FIG. 13.

FIG. 15 is a perspective view of an embodiment of the distal shaft end having the first and second wires in extended configurations.

FIG. 16 illustrates the front view of the distal shaft end of the embodiments of FIG. 15 and FIG. 17.

FIG. 17 is a perspective view of an embodiment of the distal shaft end having the first and second wires in extended configurations.

FIG. 18 is a top view of an embodiment of the distal shaft end having the first and second wires in contracted configurations.

FIG. 19 is a front view of the embodiment of FIG. 18.

FIG. 20 is a front view of an embodiment of the distal shaft end having the first and second wires in contracted configurations.

FIG. 21 is a perspective view of the embodiment of FIG. 20 having the first and second wires in expanded configurations.

FIG. 22 is a front view of the embodiment of FIG. 18 having the first and second wires in expanded configurations.

FIG. 23 is a perspective view of an embodiment of the distal shaft end having the first and second wires in an expanded configuration.

FIG. 24 is a front view of the embodiment of FIG. 23.

FIG. 25 is a perspective view of an embodiment of the distal shaft end having the first, second and third wires in expanded configurations.

FIG. 26 is a front view of an embodiments of the distal shaft end having the first, second and third wires in expanded configurations.

FIG. 27 is a perspective view of an embodiment of the distal shaft end having the first and second wires in expanded configurations.

FIG. 28 is a front view of the embodiment of FIG. 27.

FIG. 29 is a perspective view of an embodiment of the distal shaft end having the first and second wires in expanded configurations.

FIG. 30A is front view of the embodiment of FIG. 29.

FIG. 30B is front view of an embodiment of the distal shaft end having the first and second wires in expanded configurations.

FIG. 31 is a perspective view of an embodiment of the distal shaft end having a separating hoop in a contracted configuration.

FIG. 32 is a side view of the embodiment of FIG. 31.

FIG. 33 is a perspective view of the embodiment of FIG. 31 in an expanded configuration.

FIG. 34 is a side view of the embodiment of FIG. 33.

FIG. 35 is a perspective view of an embodiment of the distal shaft end having a separating hoop in a contracted configuration.

FIG. 36 is a side view of the embodiment of FIG. 35.

FIG. 37 is a perspective view of the embodiment of FIG. 35 in an expanded configuration.

FIG. 38 is a side view of the embodiment of FIG. 37.

FIG. 39 is a perspective view of an embodiment of the distal shaft end.

FIG. 40 is a front view of the embodiment of FIG. 39.

FIGS. 41 and 42 are perspective views of various embodiments of the distal shaft end.

FIG. 43 is a perspective view of an embodiment of the distal shaft end.

FIG. 44 is a front view of the embodiments of FIGS. 43, 45 and 46.

FIG. 45 is a perspective view of an embodiment of the distal shaft end.

FIG. 46 is a side view of the embodiment of FIG. 45.

FIG. 47 is a perspective view of an embodiment of the distal shaft end.

FIG. 48 is a side view of the embodiment of FIG. 47.

FIG. 49 is a perspective view of an embodiment of the distal shaft end having a cleaving tool in a contracted configuration and a severing tool in an expanded configuration.

FIG. 50 is a perspective view of an embodiment of the distal shaft end having a cleaving tool in an expanded configuration and a severing tool in an expanded configuration.

FIG. 51 is a perspective view of an embodiment of the distal shaft end having a cleaving tool in a contracted configuration and a severing tool in a contracted configuration.

FIG. 52 is a perspective view of an embodiment of the distal shaft end.

FIG. 53 is a front view of the embodiment of FIG. 52.

FIG. 54 is a perspective view of an embodiment of the distal shaft end.

FIGS. 55-57 are side cut-away views of various embodiments of the distal shaft end.

FIG. 58 is a side view of an embodiments of the distal shaft end.

FIG. 59 is a front view of the embodiment of FIG. 58.

FIG. 60 is a side view of an embodiment of the shaft tip.

FIG. 61 is a perspective view of an embodiment of the distal shaft end.

FIGS. 62-69 illustrate various embodiments of the gigli wire saw.

FIGS. 70-72 are cut-away views of various embodiments of the handle.

FIG. 73 is a side view of an embodiment of the distal shaft end.

FIGS. 74 and 75 are partial cut-away side views of an embodiment of a method of use of the tissue separation apparatus.

FIG. 76 is a cut-away front view of an embodiment of a method of use of the tissue separation apparatus.

FIGS. 77 and 78 are cut-away side views of various embodiments of a method of use of the tissue separation apparatus.

FIG. 79 is a partial cut-away side view of an embodiment of a method of use of the tissue separation apparatus.

FIG. 80 is a side view of an embodiment of a method of use of the tissue separation apparatus.

FIG. 81 is a partial cut-away side view of an embodiment of a method of use of the tissue separation apparatus.

### DETAILED DESCRIPTION

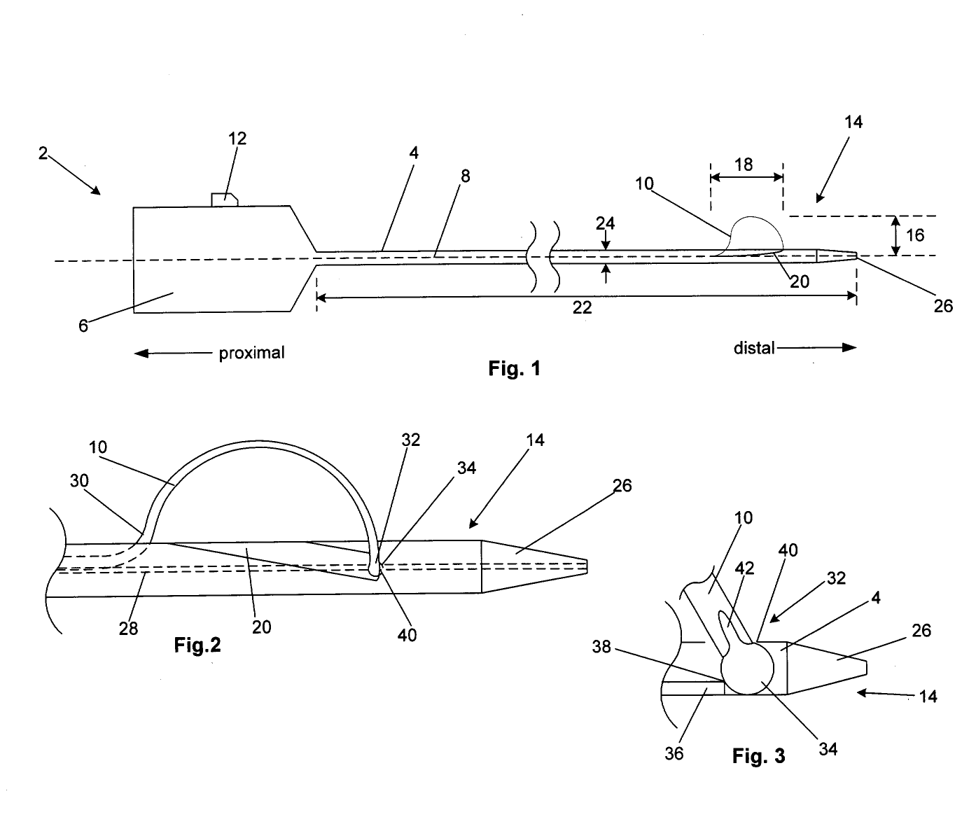

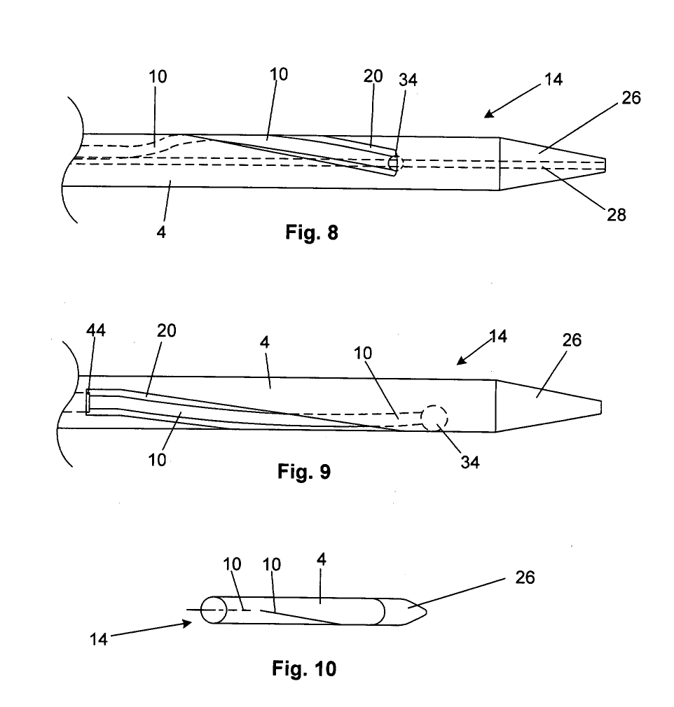

FIG. 1 illustrates an embodiment of a tissue separation apparatus 2. The apparatus 2 can have a shaft 4 (shown with a break in the length) and a handle 6. The shaft 4 can be fixedly attached to the handle 6. The shaft 4 can be rigid or resiliently flexible. The shaft 4 can be solid or hollow. A longitudinal axis 8 can be disposed along the center of the shaft 4. The shaft 4 can have a separating member, for example a substantially helical wire 10 slidably attached to the inside of the shaft 4. The handle 6 can have a control, for example a sliding switch 12, for operating the separating member. The sliding switch 12 can manipulate the wire 10 between an expanded configuration (shown in FIGS. 1-7) and a contracted configuration (shown in FIGS. 8-12). The sliding switch 12 can also manipulate the wire 10 into partially expanded and partially contracted configurations.

The wire 10 can extend radially from the longitudinal axis 8 at a distal shaft end 14. The wire 10 can be rigid or resiliently flexible. The wire 10 can have a cleaving diameter 16 that can be from about 0 cm (0 in.) to about 2.0 cm (0.79 in.), more narrowly from about 0.2 cm (0.08 in.) to about 1.2 cm (0.47 in.), for example about 1.1 cm (0.43 in.). The shaft 4 can have an extension length 18 over which the wire 10 can extend from the shaft 4. The extension length 18 can be from about from about 0 cm (0 in.) to about 50 cm (20 in.), more narrowly from about 3.0 cm (1.2 in.) to about 6.0 cm (2.4 in.), for example about 3.6 cm (1.4 in.).

The wire 10 can be made of stainless steel alloys, nickel titanium alloys (e.g., Nitinol), cobalt-chrome alloys (e.g., ELGILOY® from Elgin Specialty Metals, Elgin, Ill. CONICHROME® from Carpenter Metals Corp., Wyomissing, Pa.), polymers such as polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, silicone, radiopaque materials or a combination thereof. Examples of radiopaque materials are barium, sulfate, titanium, stainless steel, nickel-titanium alloys and gold.

The shaft 4 can have a seating groove 20. The seating groove 20 can form a helical shape on the surface of the shaft 4 over the extension length 18. The shaft 4 can have a shaft length 22 from about 10 cm (3.9 in.) to about 100 cm (39 in.), more narrowly from about 20 cm (7.9 in.) to about 60 cm (24 in.), for example about 45 cm (18 in.). The shaft 4 can also have a shaft diameter 24 from about 0.5 mm (0.02 in.) to about 4 mm (0.2 in.), for example about 1.8 mm (0.071 in.). The shaft 4 can be made of any of the materials listed for the wire 10 or a combination thereof. The shaft 4 can also be shaped as a wound wire.

FIG. 2 illustrates an embodiment of the distal shaft end 14. The distal shaft end 14 can have a shaft tip 26. The shaft tip 26 can be resilient and can be made from of any of the materials listed about for the wire 10 or a combination thereof. A guidewire channel 28 can extend longitudinally along the shaft 4 and the shaft tip 26. The wire 10 can have a proximal wire end 30 and a distal wire end 32. The distal wire end 32 can be formed into, or have added, a locking mechanism, for example a ball 34.

FIG. 3 is a cross-sectional view of an embodiment of the distal shaft end 14. The ball 34 can be rotatably attached to the shaft 4. A shelf 36 can be fixedly attached to the inside of the shaft 4. The shelf 36 can have a first fixation point 38 where the shelf 36 contacts the ball 34. The shelf 36 can cover most or all of the inside of the shaft 4, forming a partial or complete cylindrical collar or washer, thus the first fixation point 38 can be more than a single point. The side of the shaft 4 diagonally opposite the shelf 36, as shown in FIG. 3, can have a second fixation point 40 where the shaft 4 contacts the ball 34. The ball 34 can have a ball attachment structure 42. The ball attachment structure 42 can be a melted portion of the ball 34, a melted portion of the distal wire end 32, weldment, tape, glue, a screw, a bolt, a crimp, a collar or a combination thereof.

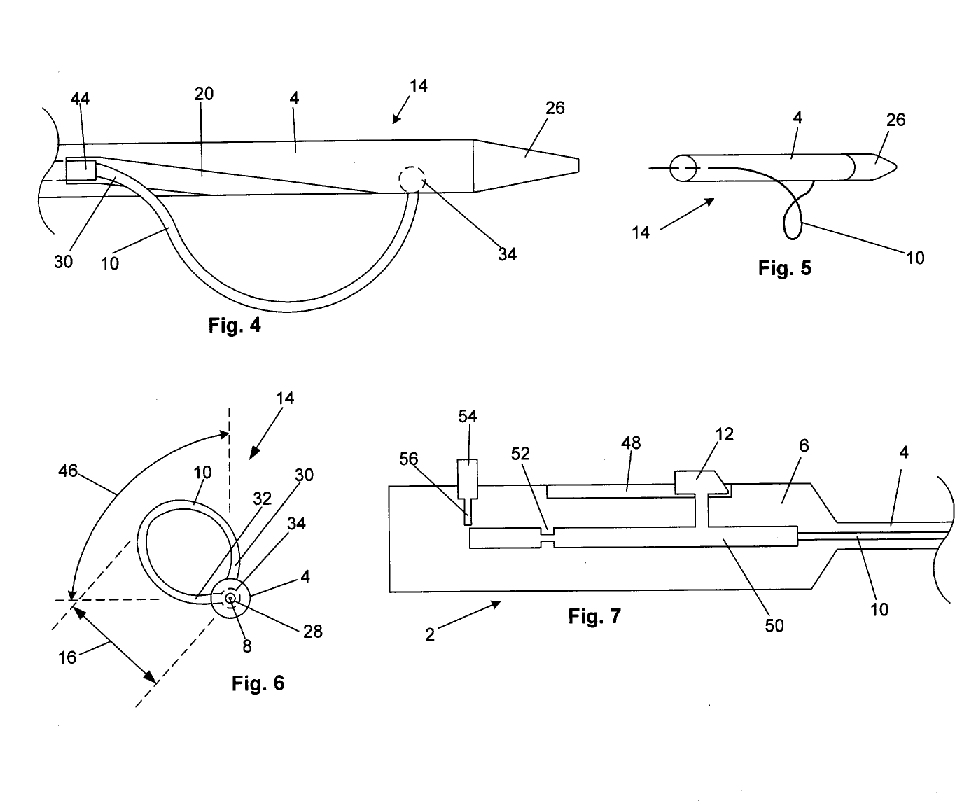

FIG. 4 illustrates an embodiment of the distal shaft end 14. The wire 10 can have a stiffened portion in the shaft 4 and a flexible portion where the wire 10 extends from the shaft 4. A stiffener 44 can be a stiffening sleeve and envelope the proximal wire end 30. The stiffener 44 can continue proximally along the wire 10 until the wire 10 proximally terminates. The stiffener 44 can be slidably or fixedly attached to the wire 10. The stiffener 44 can be a coil wrapped around the wire 10, a solid cylinder, a stiffened portion of the wire 10, for example a stiffer material or a larger diameter of wire, or a combination thereof.

FIG. 5 illustrates an embodiment of the distal shaft end 14. The wire 10 can extend from the shaft 4 and form a substantially helical shape.

FIG. 6 illustrates an embodiment of the distal shaft end 14. The wire 10 can form a substantially oval or circular shape when viewed from the front of the shaft 4. The wire 10 can form a wire extension angle 46. The wire extension angle 46 can be the angle between the distal wire end 32 and the proximal wire end 30 in the plane perpendicular to the longitudinal axis 8. The wire extension angle 46 can be from about 30° to about 360°, for example about 180°.

FIG. 7 illustrates an embodiment of the handle 6. The handle 6 can have a control track 48. The sliding switch 12 can be slidably seated in the control track 48. The sliding switch 12 can be fixedly attached to a transmission. The transmission can be a direct mechanical connection or geared to increase or decrease the movement of the wire 10 relative to the movement of the sliding switch 12. The transmission can be a rod 50. The rod 50 can be fixedly attached to the wire 10. The rod 50 can have a lock slot 52. A lock switch 54 can be slidably attached to the handle 6. The lock switch 54 can have a lock tab 56.

FIG. 8 illustrates the embodiment of the distal shaft end 14 of FIG. 2 with the wire 10 in the contracted configuration. The wire 10 can seat in the seating groove 20.

FIG. 9 illustrates the embodiment of the distal shaft end 14 of FIG. 4 with the wire 10 in the contracted configuration. The stiffener 44 can retract with the wire 10.

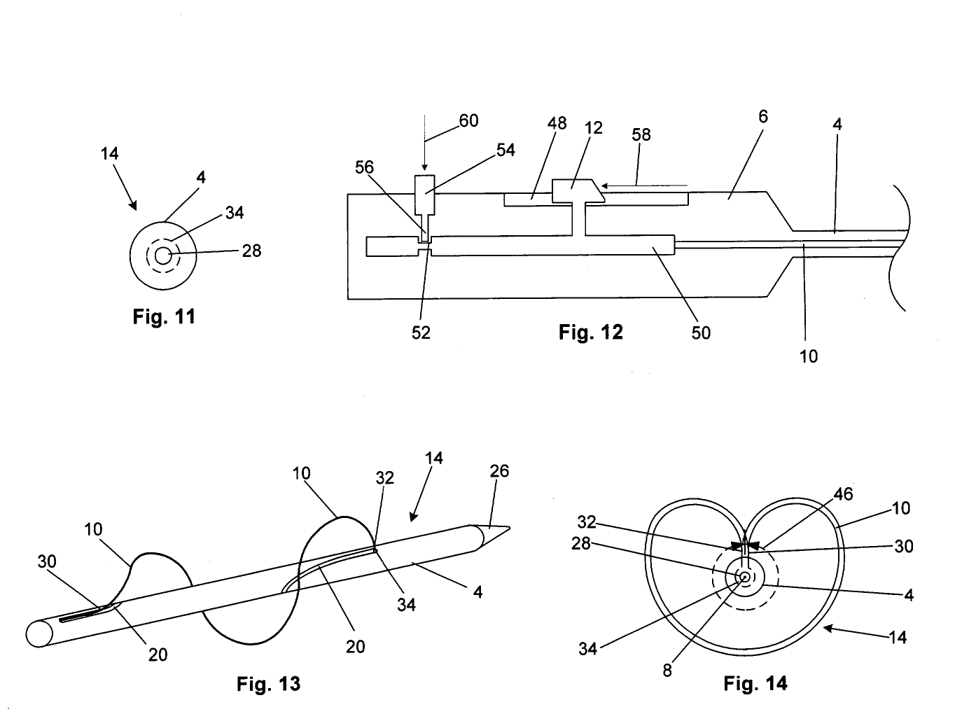

FIG. 10 illustrates the embodiment of the distal shaft end 14 of FIG. 5 with the wire 10 in the contracted configuration. FIG. 11 illustrates the embodiment of the distal shaft end 14 of FIG. 6 with the wire 10 in the contracted configuration.

FIG. 12 illustrates a cut-away view of the embodiment of the handle of FIG. 7 with the wire 10 in the contracted position. The sliding switch 12 can be moved proximally, shown by arrow 58, to retract the rod 50 and contract the wire 10. The sliding switch 12 can be moved distally to extend the rod 50 and expand the wire 10. The lock switch 54 can be depressed, shown by arrow 60, to releasably engage the lock tab 56 into the lock slot 52 and fix the position of the rod 50 and wire 10.

FIGS. 13 and 14 illustrate an embodiment of the distal shaft end 14 with the wire 10 in the extended configuration. The distal shaft end 14 can have a helically-shaped wire 10 with the wire extension angle 46 of about 360°. The distal wire end 32 and the proximal wire end 30 can substantially angularly align, as measured relative to the longitudinal axis 8.

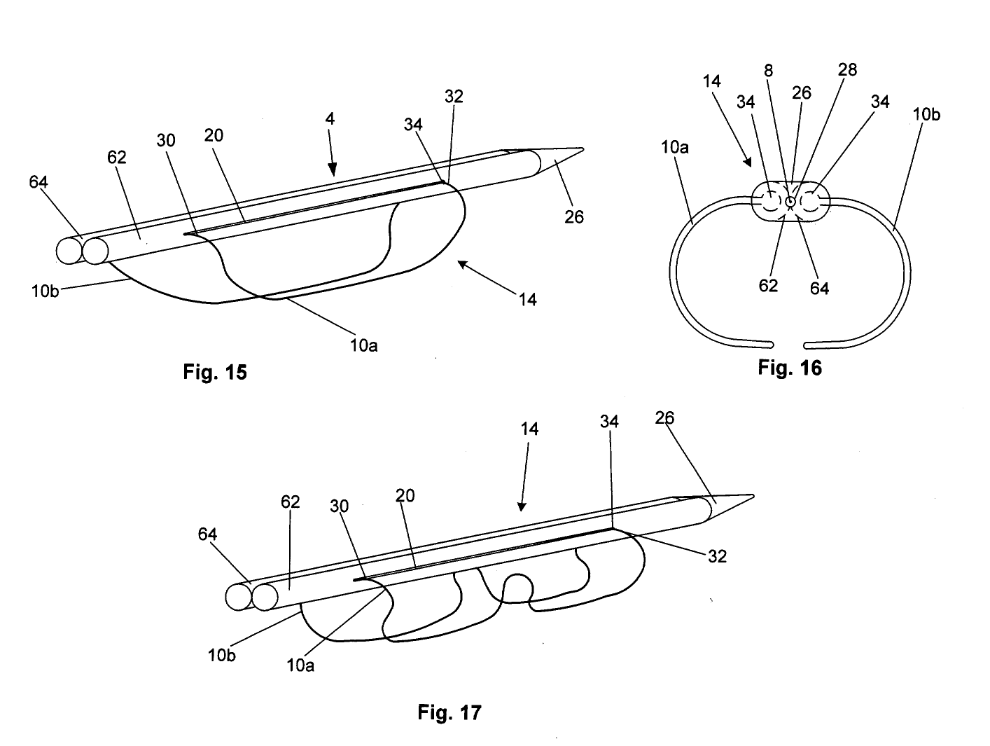

FIGS. 15 and 16 illustrate an embodiment of the distal shaft end 14 with first and second wires 10a and 10b in extended configurations. The shaft 4 can have a first shaft section 62 and a second shaft section 64. The first shaft section 62 can be fixedly attached to the second shaft section 64. The shaft sections 62 and 64 can be rigid or resiliently flexible. The shaft sections 62 and 64 can be solid or hollow. The first wire 10a can be substantially within the first shaft section 62. The second wire 10b can be substantially within the second shaft section 64. The first and second wires 10a and 10b can be attached to each other (not shown) proximal to the distal shaft end 14. The shaft tip 26 can have an integral tip fixedly attached and covering the distal ends of the first and second shaft sections 62 and 64.

The wires 10a and 10b can have bat wing configurations. The bat wing configuration can have the wire 10a and/or 10b that forms a two-dimensional shape lying on a curved plane. The plane can be semi-circularly curved as shown by the wires 10a and 10b in FIG. 16. The two-dimensional shape can be a parabola, semi-circle, or a square, rectangle or triangle without one side. The distal wire end 32 and the proximal wire end 30 can substantially angularly align, as measured relative to the longitudinal axis 8.

FIG. 17 illustrates an embodiment of the distal shaft end 14 with wires 10a and 10b that can have multiple bat wing configurations. The multiple bat wing configuration can be similar to the bat wing configuration, but with the multiple bat wing configuration can have multiple two-dimensional shapes, for example two parabolas in each wire 10a and 10b.

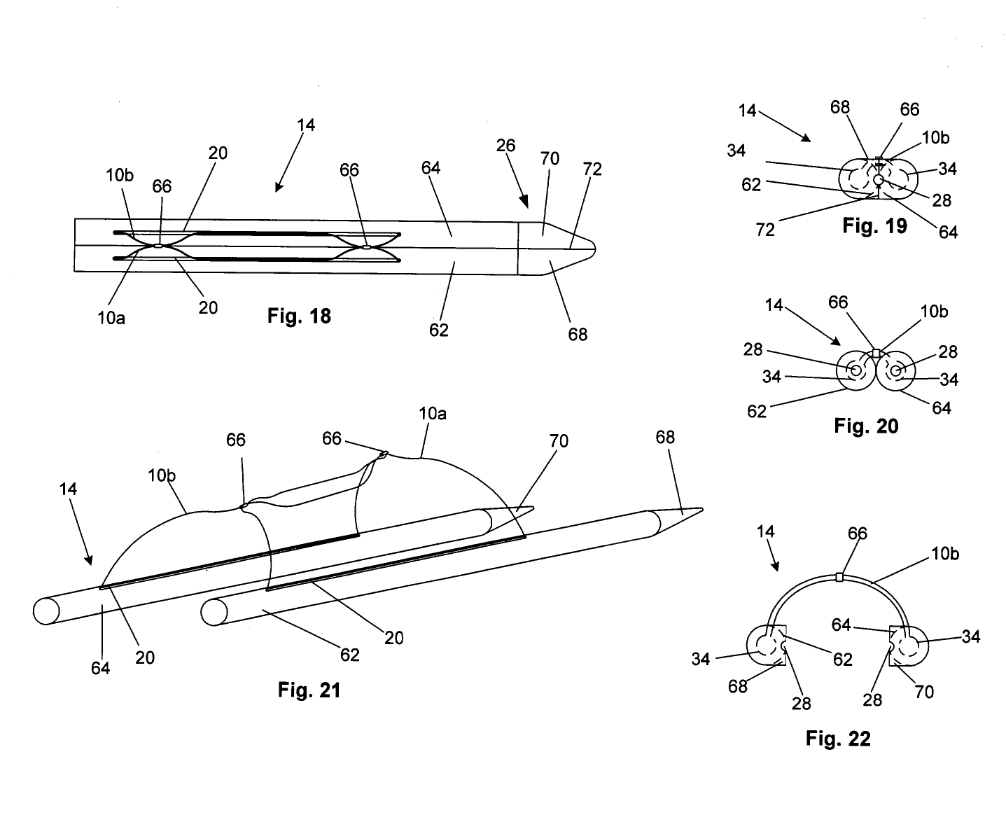

FIG. 18-20 illustrate embodiments of the distal shaft end 14 with wires 10a and 10b in contracted configurations. The wires 10a and 10b can have multiple bat wing configurations. The first wire 10a can be attached to the second wire 10b by wire attachment structures 66. The wire attachment structures 66 can be a melted portion of the wires 10a and 10b, weldment, tape, glue, a screw, a bolt, a crimp in the wires 10a and 10b, a collar or a combination thereof.

FIGS. 21 and 22 illustrate the embodiments of FIGS. 18-21 with the wires 10a and 10b in expanded configurations. The first shaft section 62 can move away from the second shaft section 64 as the wires 10a and 10b expand.

FIGS. 18, 19 and 21 illustrate an embodiment that can have the shaft tip 26 separated into a first shaft tip section 68 and a second shaft tip section 70 by a shaft tip division 72. The first shaft tip section 68 and second shaft tip section 70 can fit together to form the substantially conical shaft tip 26. FIGS. 20 and 21 illustrate an embodiment that can have a separate substantially conical shaft tip sections 68 and 70 for each shaft section 62 and 64, respectively. Each shaft section 62 and 64 can have one guidewire channel 28, shown in FIG. 21, or the distal shaft end 14 can have a single guidewire channel 28, shown in FIGS. 20 and 22.

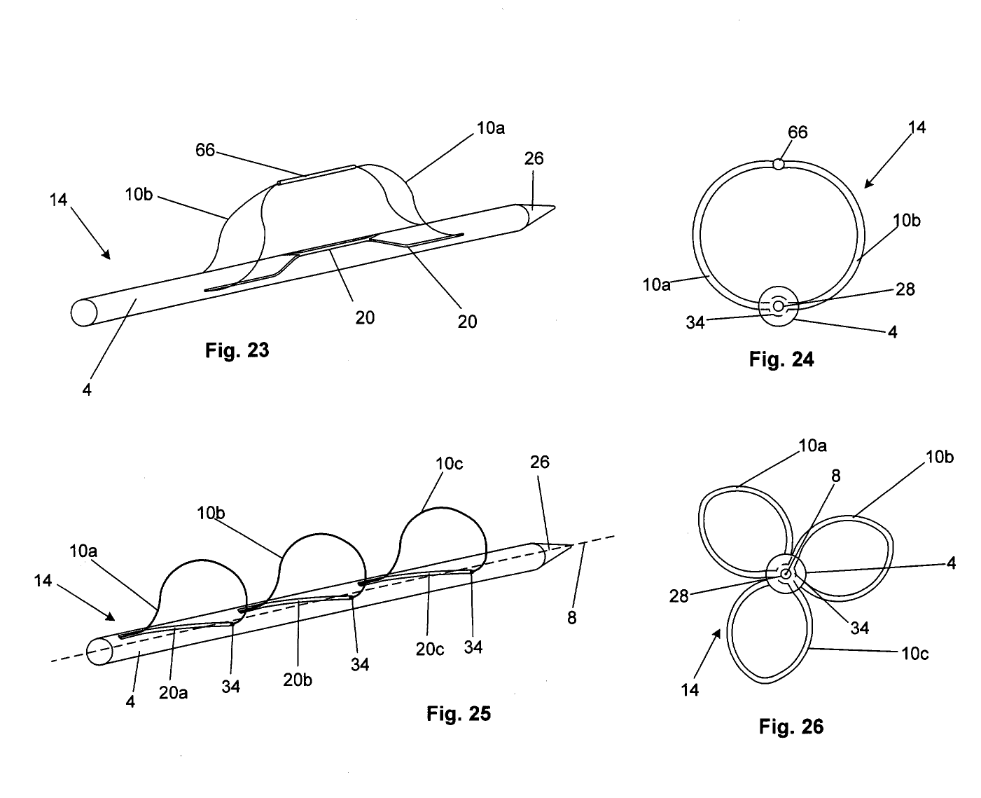

FIGS. 23 and 24 illustrate an embodiment of the distal shaft end 14 with the wires 10a and 10b in expanded configurations. The wires 10a and 10b can be formed in a bat wing configuration. The first wire 10a can be joined by the wire attachment structure 66 to the second wire 10b. The wires 10a and 10b can be attached to the shaft 4. FIGS. 25 and 26 illustrate embodiments of the distal shaft end 14 that can have a first wire 10a, a second wire 10b and a third wire 10c in expanded configurations. Each wire 10a, 10b and 10c can seat in the respective first, second and third seating grooves 20a, 20b and 20c. The wires 10a, 10b and 10c can be angularly aligned with respect to each other around the longitudinal axis 8, as shown in FIG. 25. The wires 10a, 10b and 10c can be longitudinally distributed along the shaft 4 at equal distances from the other wires 10a, 10b and 10c. As shown in FIG. 26, the wires 10a, 10b and 10c can be radially offset with respect to each other around the longitudinal axis 8, for example, the wires 10a, 10b and 10c can be angularly evenly distributed around the longitudinal axis 8, for example at increments of 120°. The wires 10a, 10b and 10c can be located at the same longitudinal position the other wires 10a, 10b and 10c along the shaft 4.

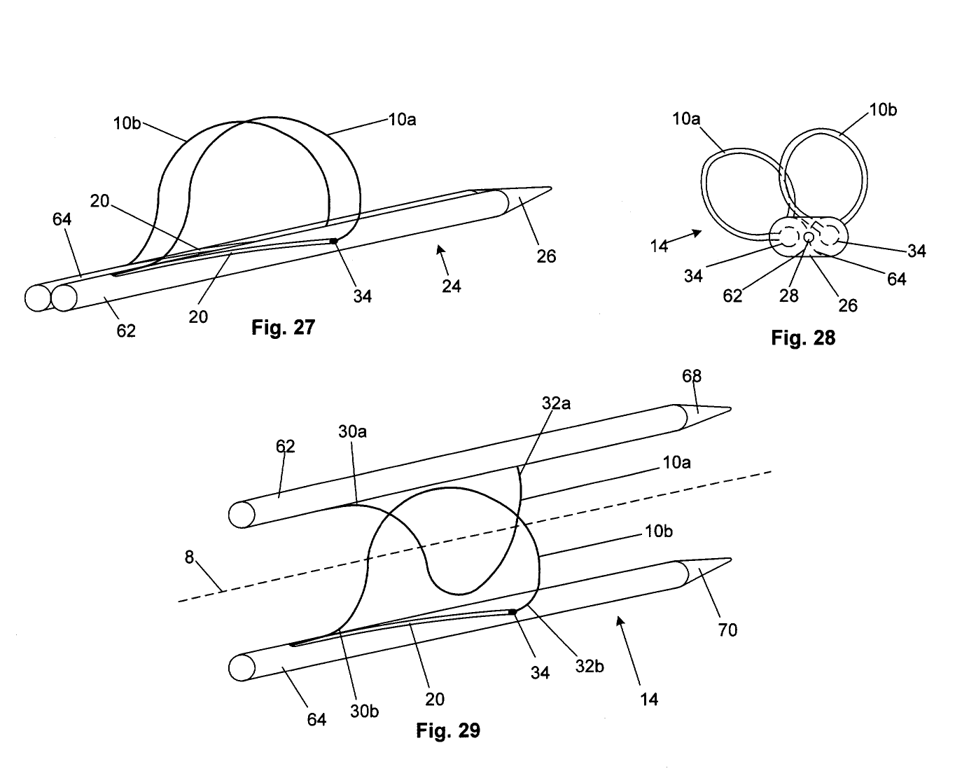

FIGS. 27 and 28 illustrate embodiments of the distal shaft end 14 that can have first and second shaft sections 62 and 64. Each shaft section 62 and 64 can have a wire 10a and 10b, respectively. Each wire 10a and 10b can be substantially angularly aligned with the other wire 10a or 10b.

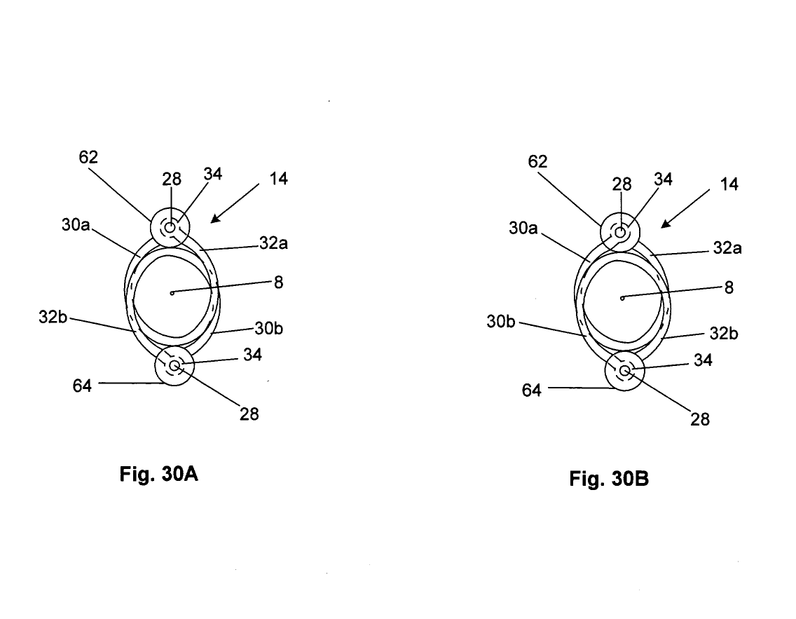

FIGS. 29, 30A and 30B illustrate embodiments of the distal shaft end 14 that can have the first shaft section 62 that can be separated from the second shaft section 64. The first shaft section 62 can be substantially parallel with the second shaft section 64 at the distal shaft end 14. The first distal wire end 32a can egress from the first shaft section 62 at an angle from the longitudinal axis 8 from about 45° to about 180°, for example about 180°, from the angle that the second distal wire end 32b can egress from the second shaft section 64. The first proximal wire end 30a can egress from the first shaft section 62 at an angle from the longitudinal axis 8 from about 45° to about 180°, for example about 180°, from the angle that the second proximal wire end 30b can egress from the second shaft section 64.

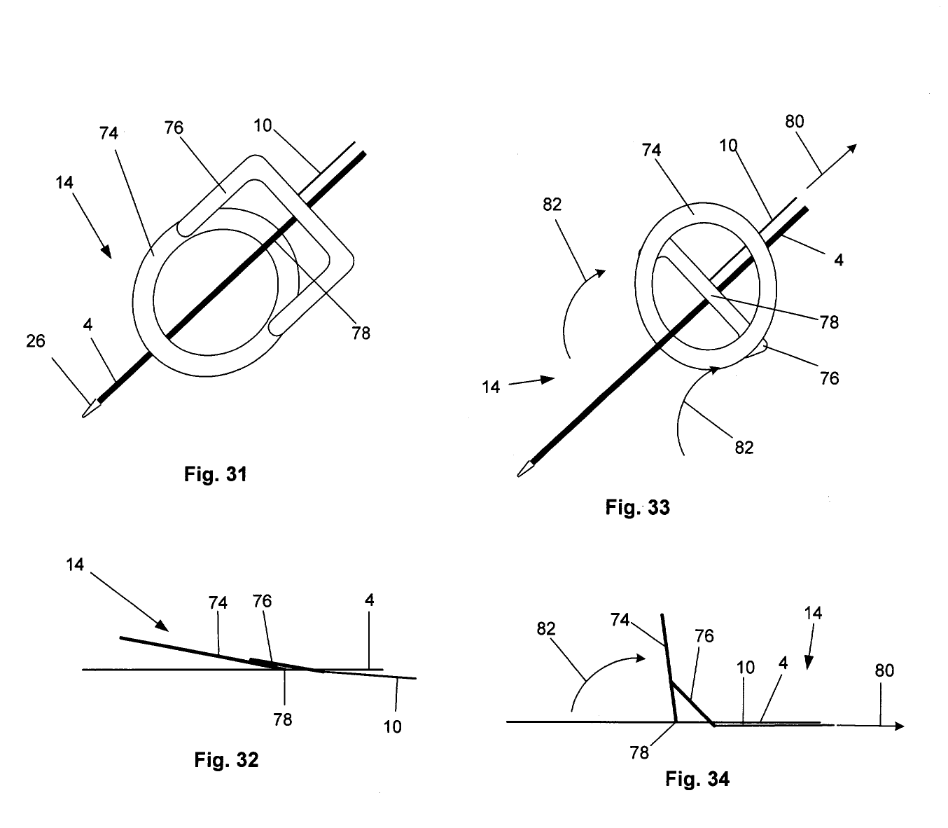

FIGS. 31 and 32 illustrate an embodiment of the distal shaft end 14 that can have a separating member that can be a separating hoop 74, for example a circular wire, shown in the contracted configuration. The separating hoop 74 can be rotatably attached to a hoop strut 76. The separating hoop 74 can also be rotatably attached to the shaft 4 at a hoop attachment point 78. The hoop strut 76 can be attached to the wire 10. The wire 10 can be inside the hollow shaft 6 until near the attachment to the hoop strut 76.

FIGS. 33 and 34 illustrate the embodiment of FIGS. 31 and 32 in an expanded configuration. The wire 10 can be proximally moved, shown by arrow 80, causing the separating hoop 74 to rotate, shown by arrow 82, with respect to the shaft 4.

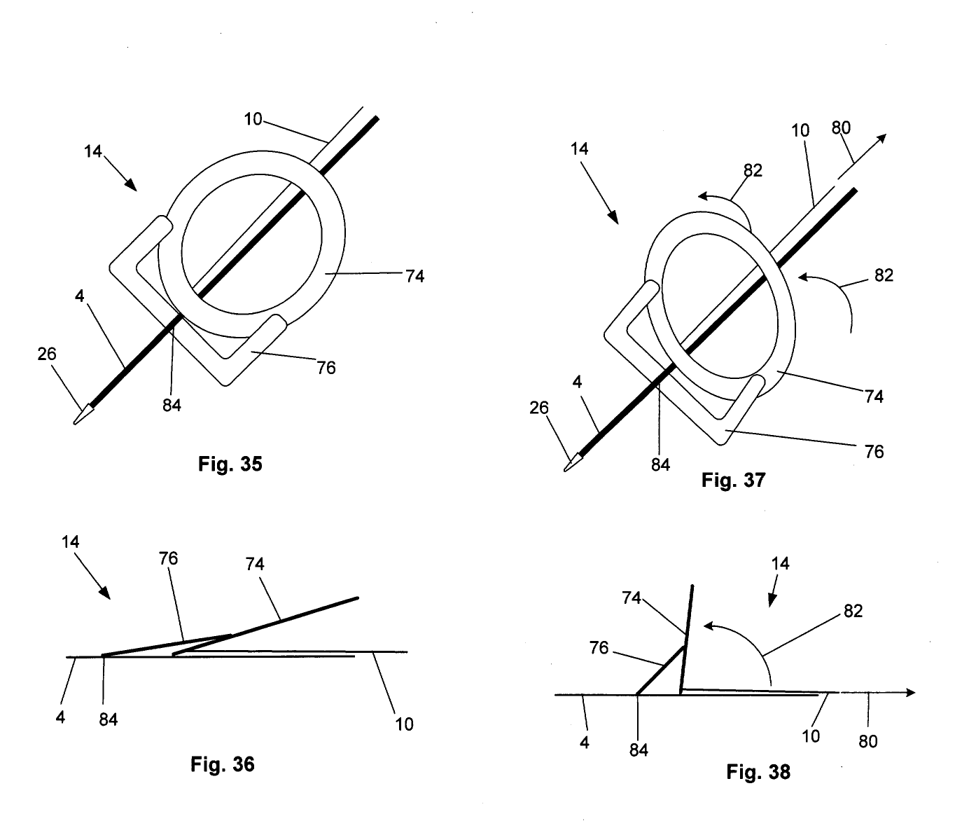

FIGS. 35 and 36 illustrate an embodiment of the distal shaft end 14 that can have the hoop strut 76 rotatably attached to the shaft 4 at a strut attachment point 84. The separating hoop 74 can be attached to the wire 10.

FIGS. 37 and 38 illustrate the embodiment of FIGS. 35 and 36 in an expanded configuration. The wire 10 can be proximally moved, shown by arrow 80, causing the separating hoop 74 to rotate, shown by arrow 82, with respect to the shaft 4.

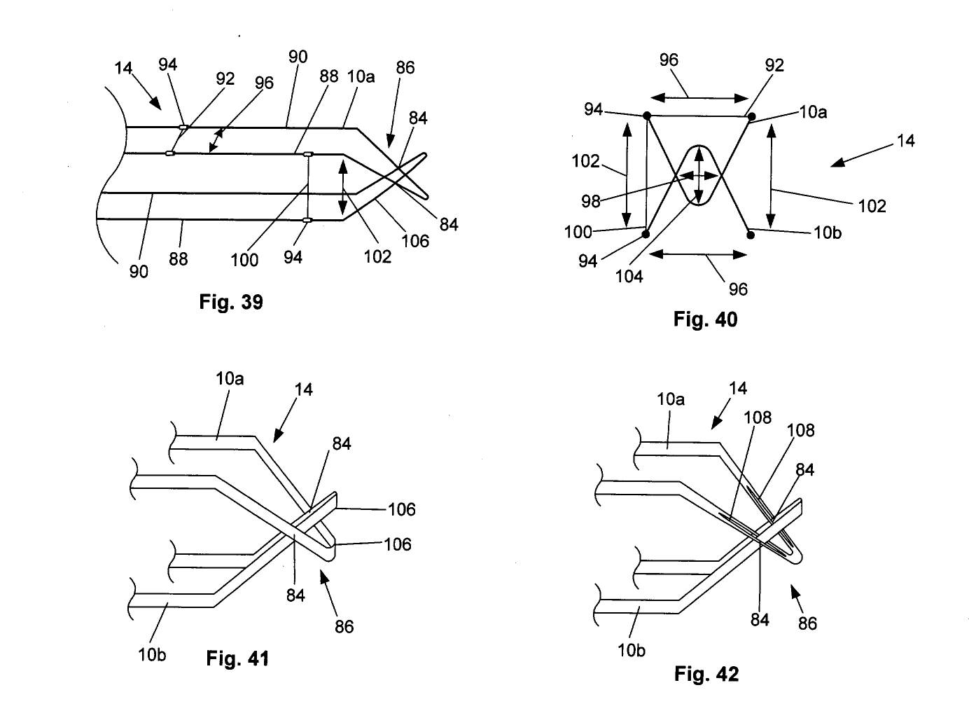

FIG. 39 illustrates an embodiment of the distal shaft end 14 that can have a jawed configuration. The jawed configuration can have the first wire 10a that can contact the second wire 10b at intersection points 84. The first wire 10a can be slidably attached to the second wire 10b at the intersection points 84. The intersection points 84 can be at a distal tip 86. The distal tip 86 can include the leading edges, or edges that can perform tissue cleaving, of the distal shaft end 14.

The wires 10a and 10b can have first arms 88 and second arms 90. A width strut 92 can by fixedly attached to sliding collars 94. The sliding collars 94 can be slidably attached to the arms 88 and 90. The size of the width strut 92 can be selected before use or adjusted during use to control the arm width distance 96. Adjustments in the arm width distance 96 can adjust a tip width distance 98. A height strut 100 can be fixedly attached to sliding collars 94. The sliding collars 94 can be slidably attached to arms 88 or 90 on the first wire 10a and the second wire 10b. The size of the height strut 100 can be selected before use or adjusted during use to control the arm height distance 102. Adjustments in the arm height distance 102 can adjust a tip height distance 104.

FIG. 41 illustrates an embodiment of the distal shaft end 14 that can have cutting edges 106 at the distal tip 86 of the wires 10a and 10b. The cutting edges 106 can be sharpened or hardened. The wires 10a and 10b can be formed into flattened blades.

FIG. 42 illustrates an embodiment of the distal shaft end 14 that can have sliding slots 108 at the distal tip 86 of the first wire 10a. The second wire 10b can pass through the sliding slots 108.

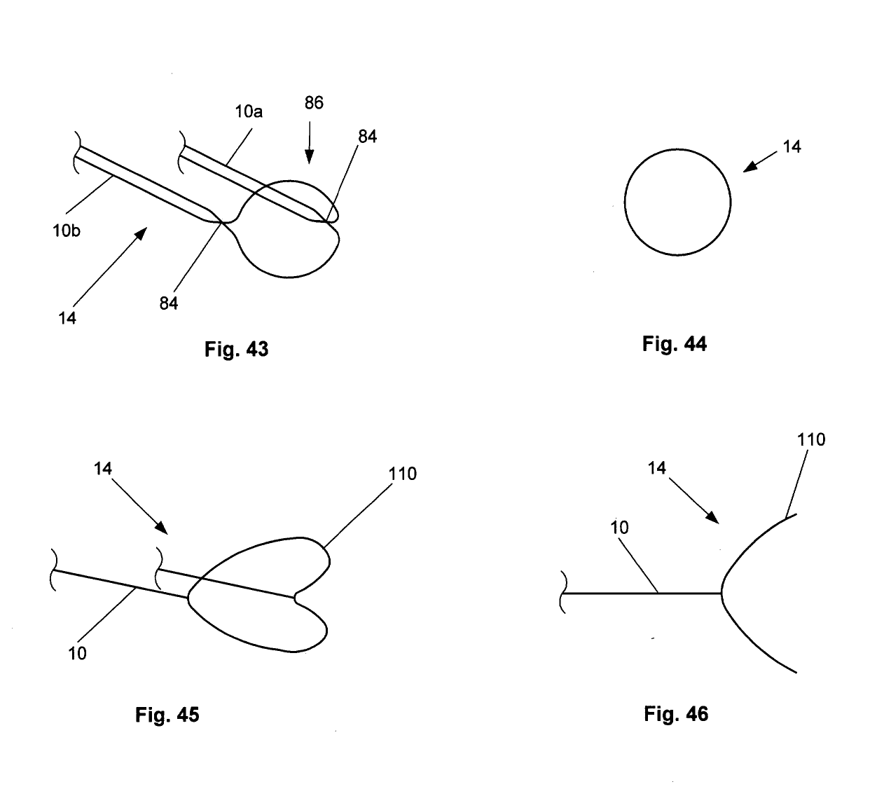

FIGS. 43 and 44 illustrate an embodiment of the distal shaft end 14 that can have a duckbill configuration. The duckbill configuration can have the first wire 10a that can slidably contact the second wire 10b at the intersection points 84 proximal to the distal tip 86. The first wire 10a can be slidably attached to, or slidably contact, the second wire 10b at the intersection points 84.

FIGS. 44, 45 and 46 illustrate an embodiment of the distal shaft end 14 that can have a mouth 110. The mouth 110 can have a two-dimensional shape lying on a curved plane. The plane can be semi-circularly or parabolically curved as shown by the side view of the mouth 110 in FIG. 46. The two-dimensional shape can be a circle, oval, square, rectangle or triangle. The mouth 110 can be resiliently flexible. The mouth 110 can be fixedly attached to, or an integral part of, the wire 10.

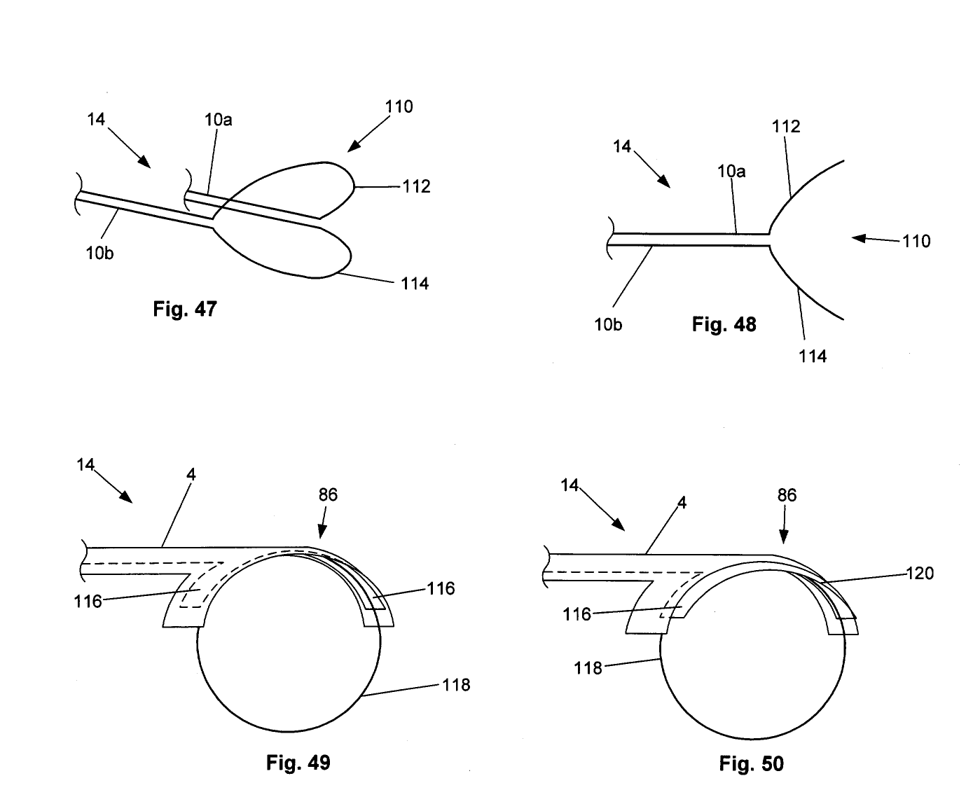

FIGS. 47 and 48 illustrate an embodiment of the distal shaft end 14 that can have a bifurcated mouth 110 having a top mouth portion 112 and a bottom mouth portion 114. The first wire 10a can form the top portion of the mouth 112. The second wire 106 can form the bottom portion of the mouth 114.

FIGS. 49 to 51 illustrate an embodiment of the distal shaft end 14 that can have a cleaving tool 116 seated in the distal tip 86. The distal tip 86 can be partially cylindrical. The distal tip 86 can also have an adjustable diameter severing tool 118. The cleaving tool 116 and the severing tool 118 can be made from any of the materials listed for the wire 10. The cleaving tool 116 can be a chisel, blade, roller, wedge or a combination thereof. The severing tool 118 can be a wire. FIG. 49 illustrates the cleaving tool 116 in the contracted configuration and the severing tool 118 in the expanded configuration.

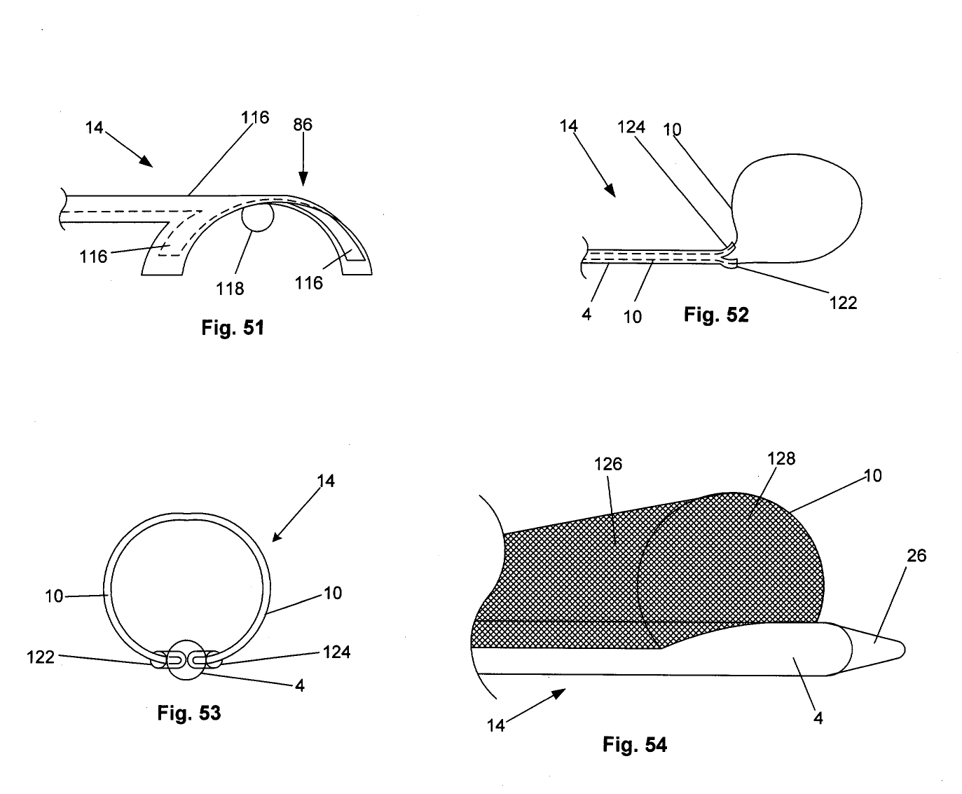

FIG. 50 illustrates the cleaving tool 116 in the expanded configuration. The cleaving tool 116 can have a cleaving edge 120. FIG. 51 illustrates the severing tool 118 in a contracted configuration.

FIGS. 52 and 53 illustrate a frameless gigli embodiment of the distal shaft end 14 that can have a looped wire 10. The shaft 4 can have first and second stiffening arms 122 and 124. The looped wire 10 can egress from the shaft 4 through the first stiffening arm 122. After the wire 10 egresses from the first stiffening arm 122, the wire 10 can form a substantially closed shape, for example, a substantially closed circle or oval, and enter the shaft 4 through the second stiffening arm 124. Both ends of the looped wire 10 can contract or expand together and/or either end of the looped wire 10 can contract or expand independent of the other end of the looped wire 10.

FIG. 54 illustrates an embodiment of the distal shaft end 14 that can have a bag 126 attached to the wire 10. The bag 126 can also be attached to the shaft 4. The bag 126 can be flexible. The bag 126 can be made from solid or mesh fabric, for example polyester (e.g., DACRON® from E. I. du Pont de Nemours and Company, Wilmington, Del.), polypropylene, PTFE, ePTFE, nylon, extruded collagen, silicone or a combination thereof. The bag 126 can have an open entryway 128. The entryway 128 can occupy the area defined by the wire 10 and the shaft 4.

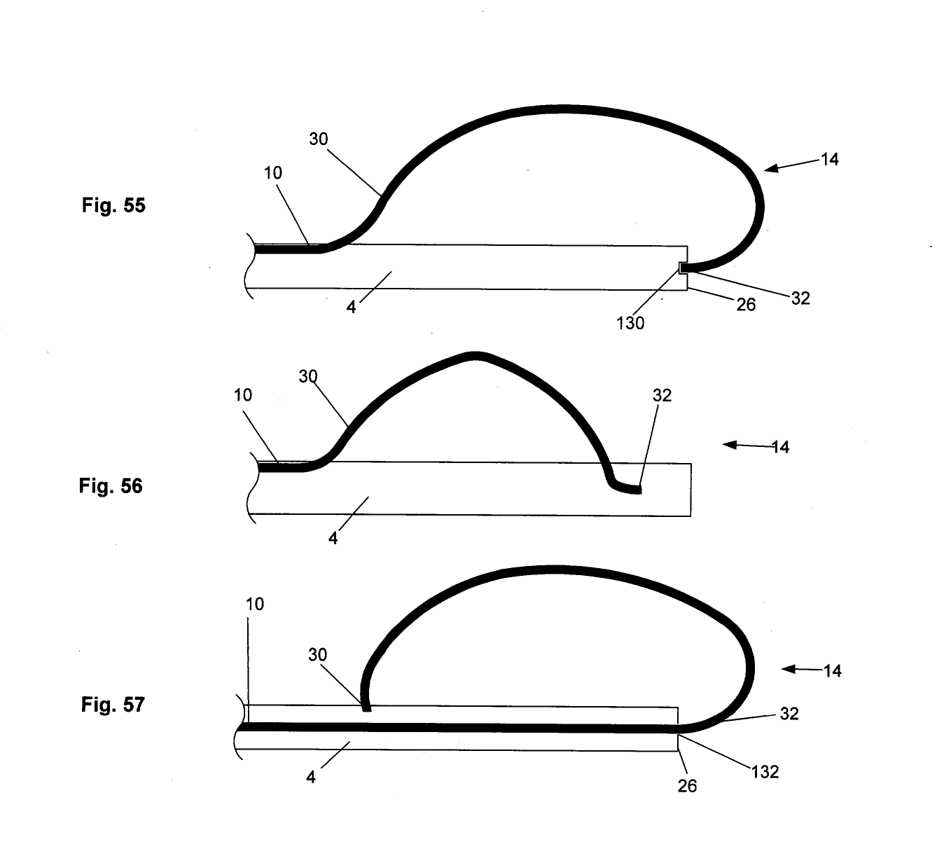

FIG. 55 illustrates an embodiment of the distal shaft end 24 that can have the distal wire end 32 terminate and be fixedly attached to the shaft tip 26. The distal wire end 32 can fixedly attach to the shaft tip 26 at a wire seat 130.

FIG. 56 illustrates an embodiment of the distal shaft end 14 that can have the distal wire end 32 terminate and be fixedly attached to the shaft 4. The proximal wire end 30 can egress from the shaft 4 through the side of the shaft 4 for the embodiments shown in FIGS. 55 and 56.

FIG. 57 illustrates an embodiment of the distal shaft end 14 that can have the proximal wire end 30 terminate and be fixedly attached to the shaft 4. The distal wire end 32 can egress from the shaft 4 through a shaft tip hole 132 in the shaft tip 26.

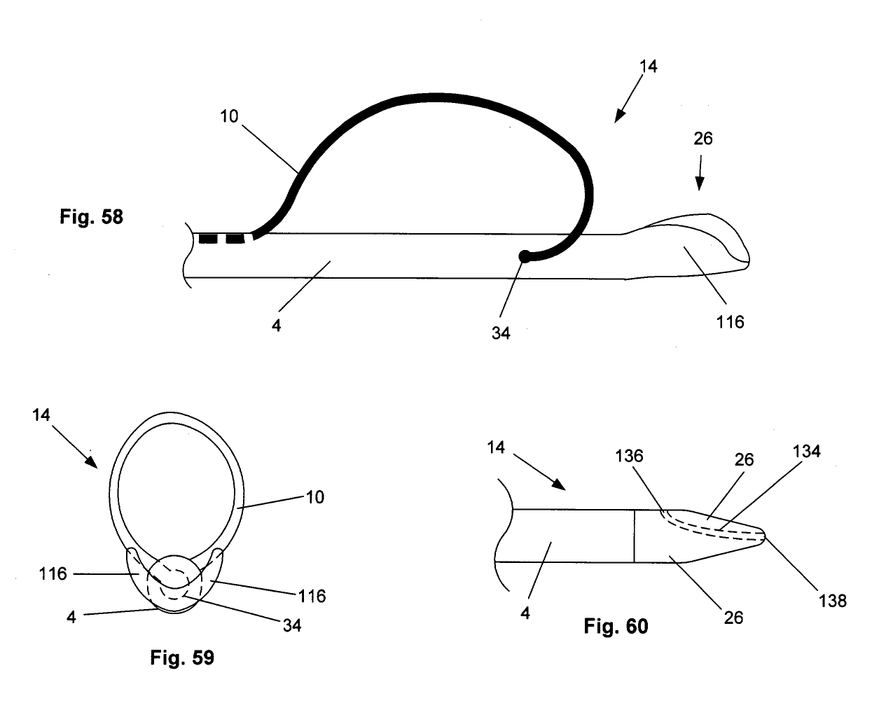

FIGS. 58 and 59 illustrate an embodiment of the distal shaft end 14 that can have a cleaving tool 116 at the shaft tip 26. The cleaving tool 116 can form a substantially semi-cylindrical shape. The cleaving tool 116 can be integrated with the shaft 4.

FIG. 60 illustrates an embodiment of the distal shaft end 14 that can have a guide-eye passage 134 in the shaft tip 26. The guide-eye passage 134 can open at a side hole 136 on the side of the shaft tip 26 and at an end hole 138 at the distal terminus of the shaft tip 26.

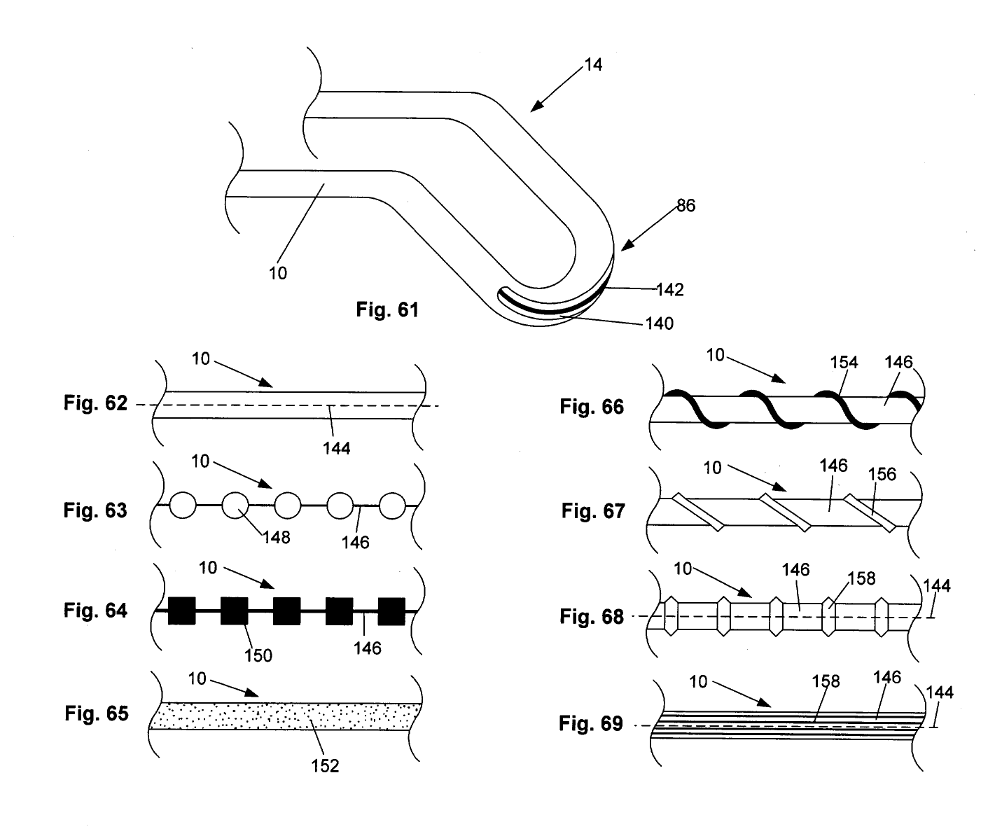

FIG. 61 illustrates an embodiment of the wire 10 that can be hollow and can have a window 140. The window 140 can be substantially hemi-circular. The window 140 can be on the distal tip 86. A gigli wire saw 142 can be inside the wire 10. The gigli wire saw 142 can be exposed through the window 140 to the outside of the wire 10. An alternative embodiment (not shown) can have the gigli wire saw 142 on the outside of the wire 10. The gigli wire saw 142 can be slidably attached to a track (not shown) in the wire 10.

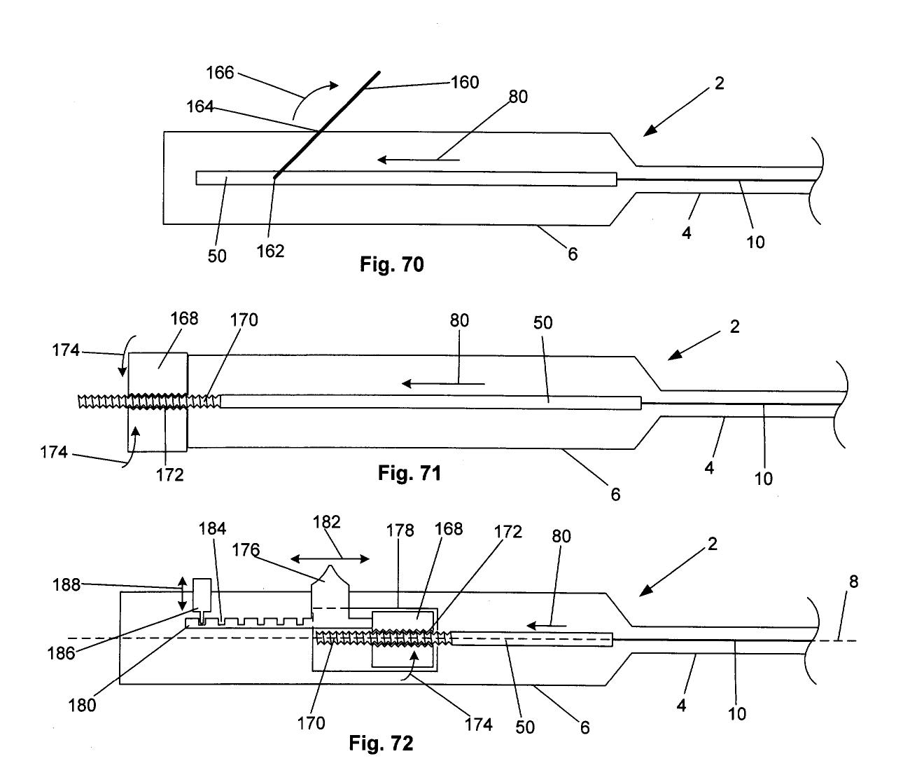

FIGS. 62 to 69 illustrate various embodiments of the gigli wire saw 142 and/or the wire 10 (referred to as wire 10 in the following descriptions for simplicity). FIG. 62 illustrates a wire 10 that can have a wire longitudinal axis 144. FIG. 63 illustrates a line 146 that can connect multiple serrating volumes, for example spherical beads 148 or cubic beads. FIG. 64 illustrates the line 146 that can connect multiple castellations 150. FIG. 65 illustrates the wire 10 that can be coated with an abrasive particulate 152, for example diamond or sand. FIG. 66 illustrates the line 146 wrapped by a coil 154. The wire 10 can be made of other combinations of braided elements, for example three braided lines 146. FIG. 67 illustrates the line 146 that can have threads 156. FIG. 68 illustrates the line 146 that can have teeth 158. The teeth 158 can be angled perpendicular to the wire longitudinal axis 144. FIG. 69 illustrates that the teeth 158 can be parallel to the wire longitudinal axis 144. The line 146, serrating volumes, castellations 150, coils 154, threads 156, and teeth 158 can be made of any of the materials listed for the wire 10 or a combination thereof.

FIG. 70 illustrates an embodiment of the handle 6 that can have a control lever 160. The control lever 160 can be rotatably attached to the rod 50 at a lever attachment point 162. The control lever 160 can egress from the handle 6 at a lever egress point 164. The control lever 160 can be rotatably and slidably attached to the handle 6 at the lever egress point 164. The portion of the control lever 160 outside of the handle 6 can be distally moved, shown by arrow 166, to cause the rod 50 and wire 10 to move proximally, shown by arrow 80.

FIG. 71 illustrates an embodiment of the handle 6 that can have a control knob 168. The control knob 168 can be rotatably attached to the handle 6. The proximal end of the rod 50 can have a threaded rod length 170. The threaded rod length 170 can be externally threaded and can threadedly attach to a threaded knob length 172. The threaded knob length 172 can be internally threaded. The control knob 168 can be rotated, shown by arrows 174, to cause the rod 50 and wire 10 to move proximally, shown by arrow 80.

FIG. 72 illustrates an embodiment of the handle 6 that can have a control knob 168 and a finite adjustment sliding switch 176. The control knob 168 can be rotated, shown by arrow 174, to proximally move the rod 50 and wire 10, shown by arrow 80. The control knob 168 can be accessed through the knob port 178. The control knob 168 can be attached to a finite adjustment rod 180. The finite adjustment rod 180 can be fixedly attached to the finite adjustment sliding switch 176. The finite adjustment sliding switch 176 can be proximally and distally moved, shown by arrows 182, to adjust the expansion and contraction of the wire 10. The finite adjustment rod 180 can have multiple finite adjustment setting slots 184. The finite adjustment setting slots 184 can be evenly spaced parallel with the longitudinal axis 8 along the finite adjustment rod 180. The handle 6 can have a finite adjustment lock 186. The finite adjustment lock 186 can be raised and lowered, shown by arrows 188, into the finite adjustment setting slots 184. The finite adjustment lock 186 can be self-actuated, for example by a spring (not shown).

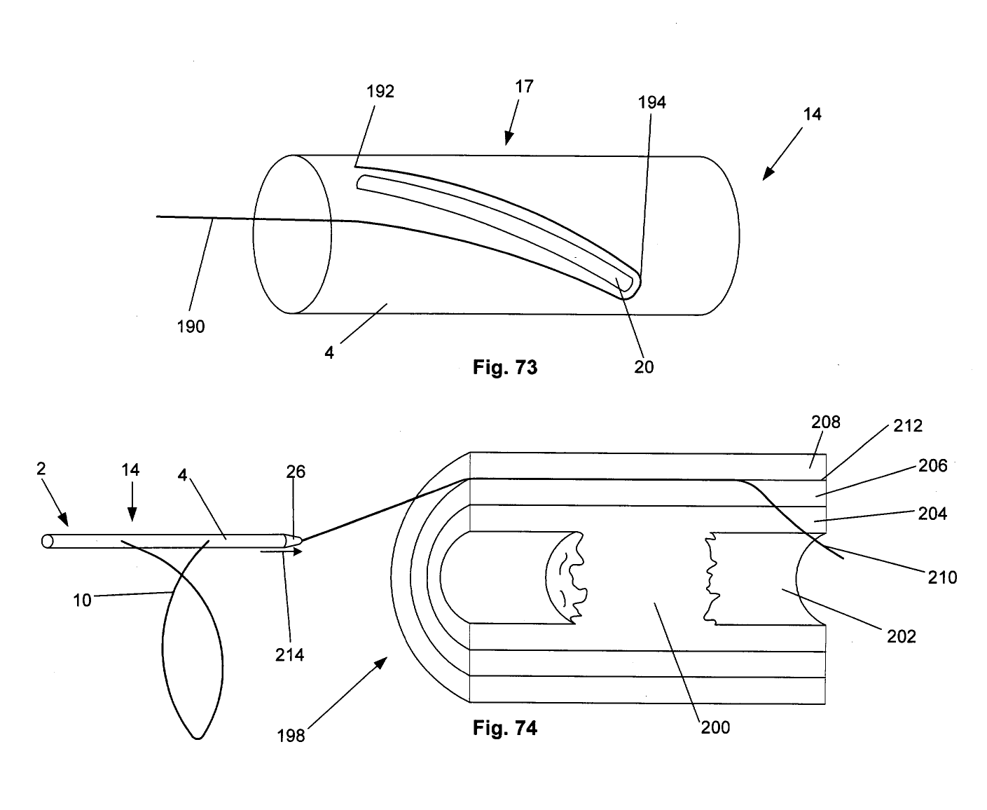

FIG. 73 illustrates an embodiment of the distal shaft end 14 that can have a garotte 190 attached to the outside of the shaft 4. The garotte 190 can be fixedly attached to the shaft 4 at a first garotte fixturing point 192. The garotte 190 can be releasably attached to the shaft 4 at a second garotte fixturing point 194. The garotte 190 can be shaped and attached to the fixturing points 190 and 192 such that the garotte 190 can surround the seating groove 20 on at least two sides. The garotte 190 can have a free end 196.

Method of Making

The wires 10a, 10b and 10c can be preformed into the desired expanded shape by methods known to those having ordinary skill in the art. The wire 10 can be slip-fit into the stiffener 44, for example a stiffening collar, shown in FIGS. 4 and 9.

The ball 34 can be an integral portion of the wire 10 that has been melted into the shape of the ball 34. The ball 34 and wire 10 can be integral and formed from a larger piece of material, for example, by stamping, melting, die cutting, laser cutting, electrical discharge machining (EDM) or a combination thereof. The ball 34 can also be secured to the end of the wire by screwing, welding, gluing,

The mouth 110, shown in FIGS. 44-48, can be formed from the wire 10. The mouth 110 can be formed separate from the wire 10 and fixedly attached to the wire 10.

The severing tool 118, shown in FIGS. 49-52 can be an extension of the internal wire 10, the severing tool 118 can be formed separate from the wire 10 and fixedly attached to the wire 10, or a combination of the manufacturing methods thereof. The cleaving edge 120 can also be sharpened and/or hardened.

The distal wire end 32, shown in FIGS. 55 and 56, and the proximal wire end 30, shown in FIG. 57, can be attached to the shaft 4 or shaft tip 26 by melting, welding, taping, gluing, screwing, bolting, crimping, collaring or a combination of methods thereof.

The releasable attachment at the second garotte fixturing point 194, shown in FIG. 73, can be made by fitting a piece of heat-shrink material around the distal shaft end 14 at the second garotte fixturing point 194. The heat-shrink material can cover the garotte 190 at the second garotte fixturing point 194, but the heat-shrink material can tear away if the free end 196 of the garotte 190 is pulled. The releasable attachment can also be made by tying a releasable knot in the garotte 190 to a post (not shown) or into a small hole (not shown) in the shaft 4 at the second garotte fixturing point 194. The releasable attachment can be a releasable mechanical catch (not shown) that can fixedly attach to the garotte 190 until the mechanical catch is manipulated to release the garotte 190.

Method of Using

FIG. 74 illustrates an embodiment of introducing the tissue separation apparatus 2 to a diseased vascular site 198. The vascular site 198 can have an occlusion 200 blocking a vascular flow pathway 202. The vascular site 198 can have a tunica intima 204, a tunica media 206 and a tunica adventitia 208. In some diseased vascular sites, the intima 204 is indistinguishable from the media 206 and/or the intima 204 is indistinguishable from the occlusion 200. A guidewire 210 can be guided into a cleaving plane 212 between the media 206 and the adventitia 208. After the guidewire 210 passes distal to the occlusion 200, the guidewire 210 can be guided into the vascular flow pathway 202. The tissue separation apparatus 2 can be moved, shown by arrow 214, with the wire 10 in an expanded state, over the guidewire 210 to the diseased vascular site 198.

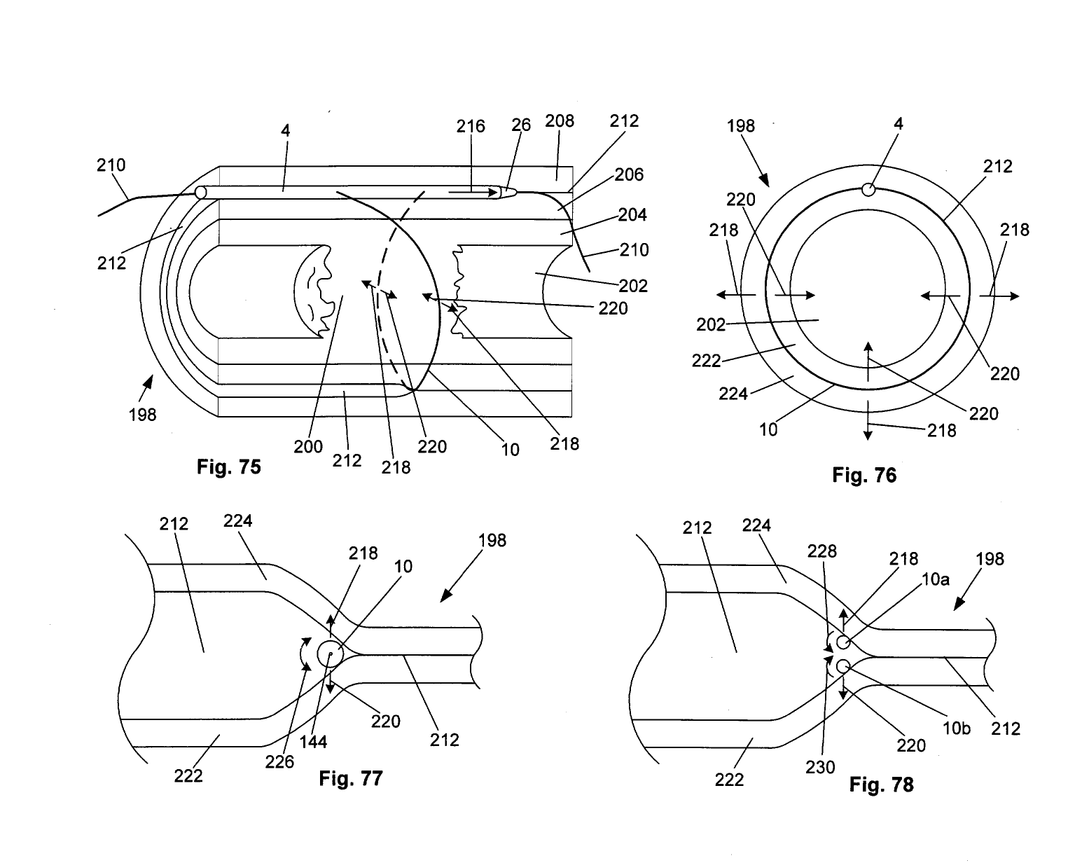

FIGS. 75 and 76 illustrate separating the cleaving plane 212. The tissue separation apparatus 2 can be moved, shown by arrow 216, through the cleaving plane 212. During the movement through the cleaving plane 212, the wire 10 can be expanded, shown by arrows 218, and contracted, shown by arrows 220, to separate inner layers 222, e.g., the intima 204 and media 206, from outer layers 224, e.g., the adventitia 208. The diseased core can be the combination of the occlusion 200 and the inner layers 222.

FIG. 77 illustrates an embodiment of separating the inner layer 222 from the outer layer 224. The wire 10 can be rotated, shown by arrow 226, about the longitudinal axis of the wire 144. The rotation of the wire 10 can pull the inner layer 222 from the outer layer 224.

FIG. 78 illustrates an embodiment of separating the inner layer 222 from the outer layer 224 using the first and second wires 10a and 10b. The first wire 10a can be placed against the outer layer 224, and the first wire 10a can be rotated in a counter-clockwise direction, shown by arrow 228. The second wire 10b can be placed against the inner layer 222, and the second wire 10b can be rotated in a clockwise direction, shown by arrow 230. The counter-revolution of the first and second wires 10a and 10b can cause the outer layer 224 to separate from the inner layer 222.

The wires 10, 10a, 10b and 10c, the separating hoop 74, the cleaving tool 116, the severing tool 118, and the gigli wire saw 142 can be connected to motors (not shown) that can create vibrating or reciprocating motion in the respective elements between at least two configurations (e.g., contracted and expanded, partially contracted and expanded, partially contracted and partially expanded). The reciprocation motion can reduce the cutting force needed, for example with hardened plaque. The motors can also cause a continuous unidirectional motion for the gigli wire saw 142 or any other continuous loop elements (e.g., several embodiments of the wire 10) used to cleave, separate or sever.

The tissue separating apparatus 2 can be actively manipulated to steer through the cleaving plane 212 and increase the separation of the inner layer 222 from the outer layer 224. The tissue separating apparatus 2 can also passively steer through the cleaving plane 212 and separate the inner layer 222 from the outer layer 224. Passive steering and separation is performed when substantially no additional force is applied to the inner layer 222 or outer layer 224 beyond the minimum force necessary to push the tissue separating apparatus 2 through the cleaving plane 212.

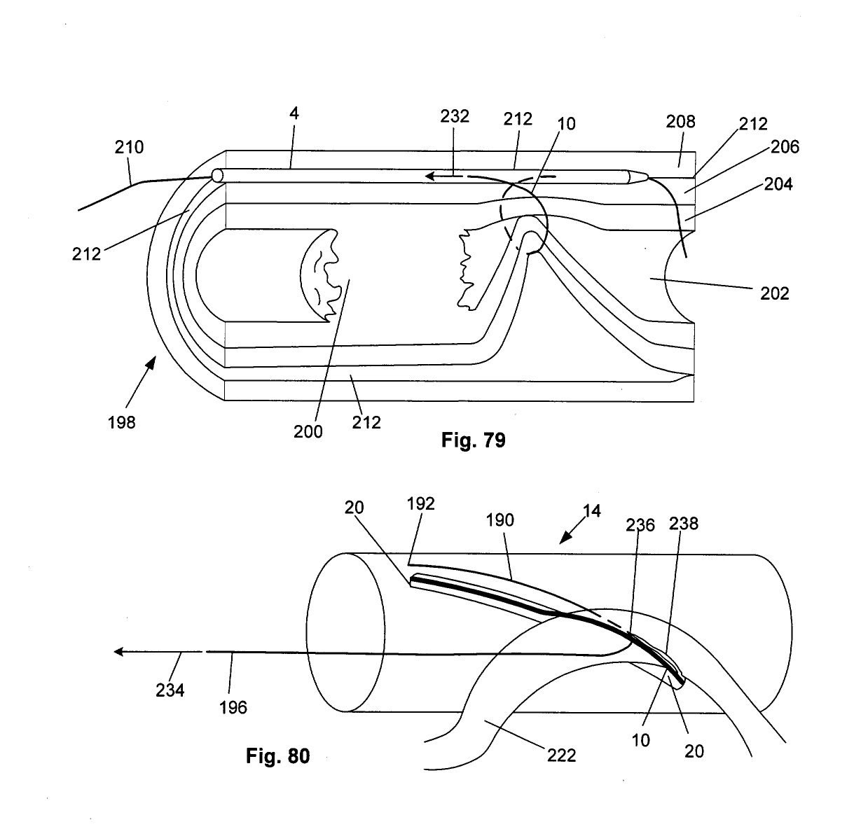

FIG. 79 illustrates an embodiment of separating the inner layer 222 from the outer layer 224 by contracting the wire 10 after the wire 10 has been moved distal to the occlusion 200. The wire 10 can be radially contracted across the vascular site 198 by retracting, shown by arrow 232, the wire 10 that extends proximal to the vascular site 198. The contraction of the wire 10 can be sufficient to pull the intima 204 and media 206 against the shaft 4.

FIG. 80 illustrates an embodiment of severing the inner layer 222 with a garotte 190. The inner layer 222 can be constrained by the wire 10 and the shaft 4. The free end 196 can be pulled, shown by arrow 234, causing the garotte 190 to release from the second garotte fixturing point 194, shown in FIG. 73, and sever the inner layer 222 at a sever point 236. The severing can create a tear 238 that can traverse the inner layer 222. The intima 204 and media 206 can be torn by the garotte 190, contracting the wire 10 tightly enough to sever the intima 204 and media 206, or a combination of methods thereof.

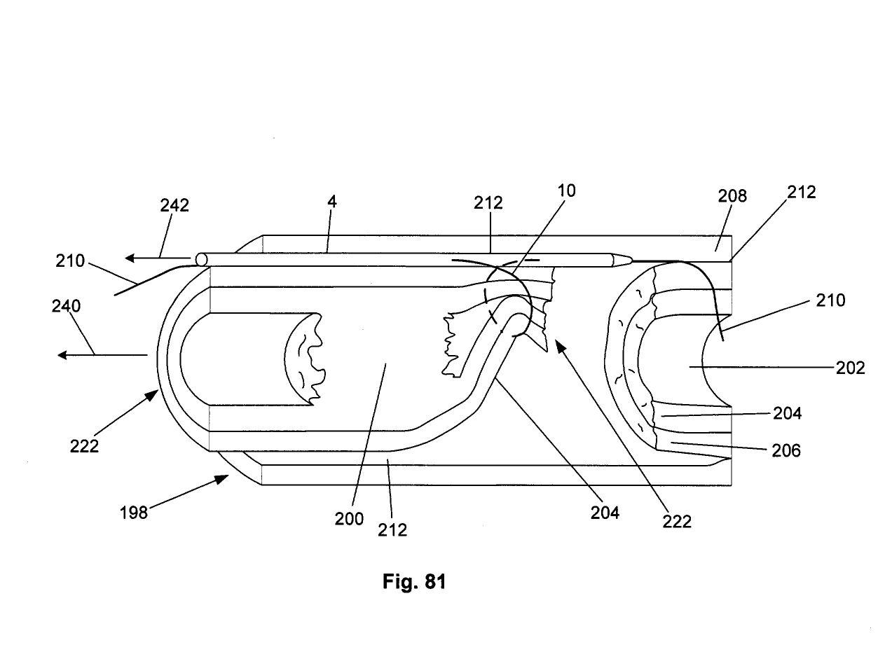

After the inner layer 222 is severed, the diseased portion of the intima 204, media 206 and occlusion 200 can be removed from the vascular site 198, shown by arrow 240, an embodiment of which is illustrated in FIG. 81. The wire 10 can secure the severed inner layer 222 against the shaft 4. The shaft 4 can be removed, shown by arrow 242, from the vascular site 198.

After the inner layer 222 has been removed, a covering or prosthetic vessel can be placed on the exposed adventitia 208. Coverings or prosthetic vessels known to those having ordinary skill in the art include grafts and stent-grafts. Stent-graft examples are provided in U.S. Pat. No. 5,879,380 to Kalmann et al. and U.S. Pat. No. 6,488,700 to Klumb et al., both of which are hereby incorporated by reference in their entirety.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

We claim:

1. An apparatus for separating tissue, the apparatus comprising:
a shaft comprising a first end, a second end and a longitudinal axis, and
a first separating member at a first length along the longitudinal axis and a first angle from the longitudinal axis,
wherein the first separating member has a contracted state and an expanded state, and wherein when placed between a first tissue layer and a second tissue layer the first separating member parts the first tissue layer from the second tissue layer in the expanded state and severs the first tissue layer in the contracted state.

2. The apparatus of claim 1, wherein the first separating member comprises a diameter from the longitudinal axis of the shaft, and wherein the diameter varies during use of the apparatus.

3. The apparatus of claim 2, wherein the diameter during the expanded state is a first diameter, and wherein the diameter during the contracted state is a second diameter, and wherein the first diameter is greater than the second diameter.

4. The apparatus of claim 1, wherein the first separating member seats into the shaft when the first separating member is in the contracted state.

5. The apparatus of claim 1, wherein the first separating member in the expanded state forms a substantially helical shape.

6. The apparatus of claim 1, wherein the first separating member in the contracted state forms a substantially helical shape.

7. The apparatus of claim 1, wherein the first separating member in the expanded state forms a substantially parabolic shape.

8. The apparatus of claim 1, wherein the first separating member in the expanded state forms a curved multi-parabolic shape.

9. The apparatus of claim 1, wherein the first separating member comprises a wire.

10. The apparatus of claim 1, further comprising a garotte.

11. The apparatus of claim 1, wherein the first end of the shaft comprises a tip.

12. The apparatus of claim 1, wherein the tip comprises a widened section.

13. The apparatus of claim 1, wherein the tip comprises a guide eye.

14. The apparatus of claim 1, wherein the first separating member is configured to actively expand and contract.

15. The apparatus of claim 1, wherein the first separating member is configured to passively expand and contract.

16. The apparatus of claim 1, further comprising a second separating member at a second length along the longitudinal axis and a second angle from the longitudinal axis.

17. The apparatus of claim 16, wherein the first length is the same as the second length.

18. The apparatus of claim 16, wherein the first angle is the same as the second angle.

19. The apparatus of claim 16, wherein the first length is greater than the second length.

20. The apparatus of claim 16, wherein the first angle is greater than the second angle.

21. A method of separating tissue comprising:
separating a first tissue layer from a second tissue layer with a member, and
severing the first tissue layer with the member, wherein the member has an expanded state and a contracted state, and wherein severing comprises contracting the member from the expanded state into the contracted state.

22. The method of claim 21, wherein severing further comprises moving a garotte through the first tissue layer.

23. The method of claim 21, further comprising inserting the member between the first tissue and second tissue layer.

24. The method of claim 21, wherein separating comprises contracting and expanding the member.

25. The method of claim 24, wherein contracting the member comprises passively contracting the member.

26. The method of claim 21, wherein expanding the member comprises passively contracting the member.

27. The method of claim 21, wherein contracting the member comprises actively contracting the member.

28. The method of claim 21, wherein expanding the member comprises actively contracting the member.
